(19) United States
(12) Patent Application Publication
(10) Pub. No.: US 2004/0057949 A1
(43) Pub. Date: Mar. 25, 2004

(54) Hemodialysis method for improving immune system function

(76) Inventors: Potito U. DePaolis, Los Angeles, CA (US); Saleh Salehmoghaddam, Los Angeles, CA (US)

Correspondence Address: OPPENHEIMER WOLFF & DONNELLY LLP, 2029 Century Park East, Suite 3800, Los Angeles, CA, 90067, US

(21) Appl. No.: 10/252,228

(22) Filed: Sep. 23, 2002

(51) Int. Cl. A61K 39/395; C02F 1/44; A61K 31/715

(52) U.S. Cl. 424/140.1; 210/647; 514/59

(57) ABSTRACT

An improved method for hemodialysis employing reverse osmosis raises the pH of the blood to preserve the activity of the enzyme lysozyme to improve immune function and reduce the risk of infection in dialysis. In general, this method comprises: (1) removing arterial blood from a patient in need of hemodialysis; (2) increasing the pH of the arterial blood to a value of about 7.8 by adding a sufficient quantity of a 5% solution of sodium chloride buffered to a pH value of 7.9; (3) passing the arterial blood from step (b) through a dialyzer operating by reverse osmosis; (4) adding a pH-reducing agent selected from the group consisting of acetate buffer and dextran sulfate to reduce the pH of the blood to its normal value; (5) passing the blood from step (4) through a dialyzer operating by reverse osmosis to remove antigen; and (6) returning the blood to the patient.

### BACKGROUND OF THE INVENTION

1. Field of the Invention

The invention is directed to methods for improving the immune system in patients treated by hemodialysis.

2. General Background and State of the Art

About 300,000 persons in the USA receive some sort of dialysis. Typically, dialysis centers, where dialysis is administered, use a hollow fiber artificial kidney to remove solutes (usually toxins), through membrane pores. Dialysates contain blood ionic substances. Excess ions diffuse down a concentration gradient until equilibrium is reached. A general description of dialysis is given in A. R,. Nissenson & R. N. Fine, “Dialysis Therapy,” (3d ed., Hanley & Belfus, Inc., Philadephia, Pa., 2002), incorporated herein by this reference.

Infections are one of the most important complications of hemodialysis. Infections are particularly prevalent in patients who have underlying disease states that leave them with increased susceptibility to infection, such as diabetes or immunodeficiency. Many infections are catheter-related.

The high concentration of adenosine and its metabolites during hemodialysis may contribute to the dialysis-induced immune deficiency, altering lymphocyte function.

### INVENTION SUMMARY

An improved method for hemodialysis employing reverse osmosis raises the pH of the blood to preserve the activity of the enzyme lysozyme to improve immune function and reduce the risk of infection in dialysis. In general, this method comprises: (1) removing arterial blood from a patient in need of hemodialysis; (2) increasing the pH of the arterial blood to a value of about 7.8 by adding a sufficient quantity of a 5% solution of sodium chloride buffered to a pH value of 7.9; (3) passing the arterial blood from step (b) through a dialyzer operating by reverse osmosis; (4) adding a pH-reducing agent selected from the group consisting of acetate buffer and dextran sulfate to reduce the pH of the blood to its normal value; (5) passing the blood from step (4) through a dialyzer operating by reverse osmosis to remove antigen; and (6) returning the blood to the patient.

Preferably, the length of the dialyzer is increased from its normal length of 18 inches to a length of between about 24 inches to about 48 inches. More preferably, the length of the dialyzer is about 36 inches.

Typically, the concentration of lysozyme in the blood is increased.

Typically, about 80 ml of blood is drawn per minute through the dialyzers.

Typically, the resistance of the patient to infections is increased.

### DETAILED DESCRIPTION OF THE PREFERRED EMBODIMENTS

The present invention provides an improved hemodialysis method to enhance the activity of lysozyme in order to reduce the occurrence of infection.

It has been recognized for many years that the failure of bacteria to grow in egg albumin is due to the presence of a substance with germicidal activity, lysozyme. The lytic principle in egg albumin is an enzyme, lysozyme, also known as muramidase. Lysozyme is usually present in many body fluids and secretions including tears, saliva, blood serum, sputum, and nasal mucus. Lysozyme has a molecular weight of about 15,000 daltons. It occurs in lysosomes intracellularly and in most extracellular fluids, especially in exocrine secretions. It is bactericidal by virtue of its lytic activity, as it hydrolyzes muramic acid, a structural element in the bacterial cell wall. It is produced by granulocytes, neutrophils, and monocytes, but not by lymphocytes. Neutrophils and monocytes are actively phagocytic, which means that they can ingest bacteria and dispose of dead cells. Once they reach the site of infection, they release certain enzymes, including lysozyme, to destroy harmful bacteria.

Serum and urine levels of lysozyme are increased in most myeloproliferative diseases. Elevated lysozyme levels occur in acute myelocytic leukemia, multiple myeloma, and renal disease. In monocytic leukemia, lysozyme is normally 0.36-0.78 mg/dl of serum, and urinary excretion is 1.3-3.6 mg/dl.

The therapeutic effectiveness of lysozyme is actually based on its ability to control the growth of susceptible bacteria and to modulate host immunity against infections and depression of immune responses. Studies (Sava-Pharmacological aspects and therapeutic applications of lysozyme) indicate that lysozyme was effective against bacterial and viral infections and also showed antiphlogistic activity in a number of pathological conditions. Radiation therapy benefited from treatment with lysozyme and much data stressed the role of lysozyme interaction with the immune system. Osserman et al. (1973) observed that human lysozyme can modify membranes and consequently mediate anti-tumor functions of macrophages. Human lysozyme has been shown to be endowed with enzymatic activity on eukaryotic cells and to modify cell immunogenicity (Warren et al. (1981).

After more than 70 years, lysozyme seems to have established its therapeutic potency in the treatment of diseases associated with a decline in immune response. It has been shown that it has a natural role and a normal turnover in the biological fluids associated with the maintenance of the control of bacterial and viral infections via its direct enzymatic activity and activation of immunological surveillance.

Aspects of the use and activity of lysozyme are disclosed in the following U.S. patents, all of which are incorporated herein by reference: U.S. Pat. No. 5,712,247 to Wu et al.; U.S. Pat. No. 5,840,294 to Kiselevsky et al.; U.S. Pat. No. 5,858,326 to Kisilevsky et al., U.S. Pat. No. 5,972,328 to Kisilevsky et al.; U.S. Pat. No. 6,099,730 to Ameer et al.; U.S. Pat. No. 6,268,164 to Lal et al.; U.S. Pat. No. 6,329,356 to Szarek et al., and U.S. Pat. No. 6,337,350 to Rahbar et al.

The stability of lysozyme is affected by the pH. Forms of lysozyme crystal were obtained by Alterton and Field at a pH between 7 and 11. Therefore, control of the pH is important in improving the stability of lysozyme.

In reverse osmosis multi-component electrolyte solutions, the concentrations of H⁺ and OH⁻ ions are of great importance. For this reason, I propose raising the pH of the blood by introducing a buffered solution of sodium chloride.

In general, a method according to the present invention comprises:

(1) removing arterial blood from a patient in need of hemodialysis;

(2) increasing the pH of the arterial blood to a value of about 7.8 by adding a sufficient quantity of a 5% solution of sodium chloride buffered to a pH value of 7.9;

(3) passing the arterial blood from step (b) through a dialyzer operating by reverse osmosis;

(4) adding a pH-reducing agent selected from the group consisting of acetate buffer and dextran sulfate to reduce the pH of the blood to its normal value;

(5) passing the blood from step (d) through a dialyzer operating by reverse osmosis to remove antigen; and

(6) returning the blood to the patient.

Preferably, the length of the dialyzer is increased from its normal length of 18 inches to a length of between about 24 inches to about 48 inches. More preferably, the length of the dialyzer is about 36 inches.

Typically, the concentration of lysozyme in the blood is increased.

Typically, about 80 ml of blood is drawn per minute through the dialyzers.

Typically, the resistance of the patient to infections is increased.

Further details are given in A. R,. Nissenson & R. N. Fine, “Dialysis Therapy,” (3d ed., Hanley & Belfus, Inc., Philadephia, Pa., 2002), incorporated herein by this reference.

While the specification describes particular embodiments of the present invention, those of ordinary skill can devise variations of the present invention without departing from the inventive concept.

We claim:

1. A method for performing hemodialysis comprising the steps of:
(a) removing arterial blood from a patient in need of hemodialysis;
(b) increasing the pH of the arterial blood to a value of about 7.8 by adding a sufficient quantity of a 5% solution of sodium chloride buffered to a pH value of 7.9;
(c) passing the arterial blood from step (b) through a dialyzer operating by reverse osmosis;
(d) adding a pH-reducing agent selected from the group consisting of acetate buffer and dextran sulfate to reduce the pH of the blood to its normal value;
(e) passing the blood from step (d) through a dialyzer operating by reverse osmosis to remove antigen; and
(f) returning the blood to the patient.

2. The method of claim 1 wherein the length of the dialyzer is increased to a length of from about 24 inches to about 48 inches.

3. The method of claim 2 wherein the length of the dialyzer is about 36 inches.

4. The method of claim 1 wherein the concentration of lysozyme in the blood is increased.

5. The method of claim 1 wherein about 80 ml of blood is drawn per minute through the dialyzers.

6. The method of claim 1 wherein the resistance of the patient to infections is increased.
